(19) United States
(12) Patent Application Publication
(10) Pub. No.: US 2013/0188775 A1
(43) Pub. Date: Jul. 25, 2013

(54) X-RAY DIAGNOSIS DEVICE

(71) Applicants: Kabushiki Kaisha Toshiba, Tokyo (JP); Toshiba Medical Systems Corporation, Otawara-shi (JP)

(72) Inventor: Yasunori GOTO, Takanezawa-machi (JP)

(73) Assignees: Toshiba Medical Systems Corporation, Otawara-shi (JP); Kabushiki Kaisha Toshiba, Tokyo (JP)

(21) Appl. No.: 13/746,839

(22) Filed: Jan. 22, 2013

(30) Foreign Application Priority Data
Jan. 23, 2012 (JP) 2012-011162

(51) Int. Cl. A61B 6/12

(52) U.S. Cl. CPC A61B 6/12
USPC 378/62

(57) ABSTRACT

An X-ray source irradiates X-rays towards the test subject. The X-ray detector detects the intensity of the X-rays penetrating the test subject. The image data generator generates X-ray images based on the intensity of the X-rays detected using the X-ray detector. Moreover, the X-ray diagnosis device comprises a revision subject image detector and an image processor. The revision subject image detector detects the image of the object of the image shaped based on the shape of the object from among the X-ray images as the image subject to revision. An image processor processes images with respect to the image subject to revision such that the difference between the luminosity value of the image subject to revision and the luminosity value of the area adjacent to the image subject to revision becomes a specified value or less.

### CROSS-REFERENCE TO RELATED APPLICATION

This application is based upon and claims the benefit of priority from Japanese Patent Application No. 2012-011162, filed on Jan., 23, 2012; the entire contents of which are incorporated herein by reference.

### FIELD

The embodiment of the present invention relates to the technology of an X-ray diagnosis device.

### BACKGROUND

The X-ray diagnosis device irradiates X-rays onto patients from X-ray tubes, captures X-rays penetrating the test subject using an X-ray detector, etc., and generates an X-ray image, which is a shadowgram proportional to the transit dose thereof. Subsequently, doctors and/or operators such as laboratory technicians, etc. (hereinafter, simply referred to as “an operator”) diagnose the test subject by investigating the X-ray images generated by the X-ray diagnosis device.

When imaging the test subject using the X-ray diagnosis device, objects other than the test subject must be imaged together with the test subject. For example, newborn infants or premature infants placed in a couveuse (an incubator) in the NICU (Neonatal Intensive Care Unit), etc. may be imaged together with the couveuse. In such cases, for example, when the couveuse surface with an open hole is included upon photographing, the X-rays attenuate because the hole penetrates the couveuse; however, the X-rays are not inhibited by the hole. Accordingly, the luminance of the hole becomes higher than the luminance of the other areas, generating a hole-shaped artifact.

Moreover, as another example, an object with a known length, referred to as a calibrated object, may be photographed together with the test subject as an index of the distance during X-ray imaging. The image of the calibrated object photographed in the X-ray image becomes unnecessary once calculation of the distance in the X-ray image is completed.

In this manner, when photographing objects other than the test subject together with the test subject, there are cases in which the artifacts generated by means of the object and the image of the object are projected upon X-ray imaging. The artifacts and images may inhibit the interpretation of the radiogram, necessitating that revision different from that of other images be carried out on the images from X-ray imaging.

### BRIEF DESCRIPTION OF THE DRAWINGS

FIG. 1 is a schematic image of the X-ray diagnosis device related to the present embodiment.

FIG. 2 is a block diagram of the X-ray diagnosis device related to the present embodiment.

FIG. 3A is a diagram showing an example of the X-ray image.

FIG. 3B is a diagram showing an example of the X-ray image.

FIG. 4 is a flow chart showing a series of actions of the X-ray diagnosis device related to the present embodiment.

### DETAILED DESCRIPTION

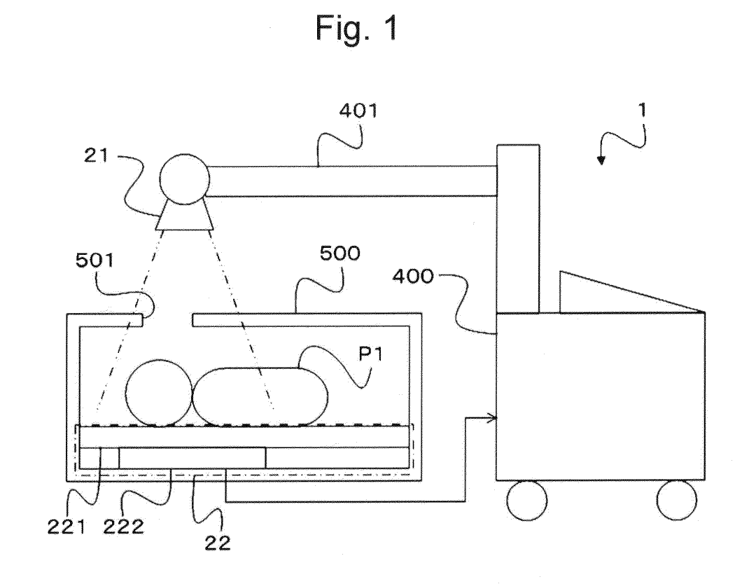

The schematic configuration of the X-ray diagnosis device related to the present embodiment is described with reference to FIG. 1. The X-ray diagnosis device related to the present embodiment includes a device body 400, an arm 401, an X-ray generating unit 21, and a photography platform 22.

The arm 401 is a retention part that retains the X-ray generating unit 21 in a predetermined position. One end of the arm 401 is fixed to the device body 400, while the X-ray generating unit 21 is retained at the other end. The X-ray generating unit 21 is configured to irradiate X-rays towards the predetermined irradiation domain. In the X-ray diagnosis device, X-ray photography is carried out by arranging the photography platform 22 in a predetermined position (hereinafter, referred to as “an exposure position” within the irradiation domain of the X-rays by means of the X-ray generating unit 21. The photography platform 22 is configured by comprising a top plate 221 and an X-ray detector 222. When the photography platform 22 is arranged in the exposure station, the platform is arranged such that the X-ray generating unit 21 and the X-ray detector 222 face each other. Moreover, the top plate 221 to be placed on a test subject P1 is interposed between the X-ray generating unit 21 and the X-ray detector 222. That is, during X-ray photography, the X-ray generating unit 21 irradiates X-rays towards the test subject P1 placed on the top plate 221. The X-ray detector 222 detects the X-rays irradiated from the X-ray generating unit 21.

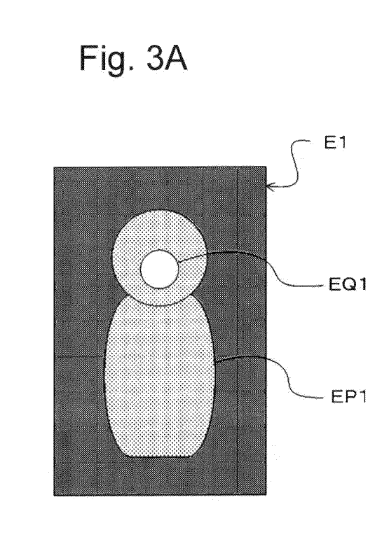

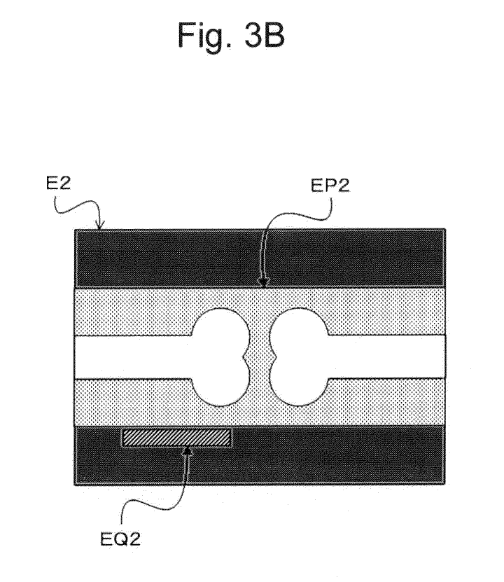

The X-ray diagnosis device related to the present embodiment assumes, for example, a case of irradiating the test subject P1 together with an object different from the test subject P1. As a specific example, as shown in FIG. 1, a test subject P1 placed in a couveuse 500 may be photographed together with the couveuse 500. An opening 501 is provided on the top of the couveuse 500. As shown in FIG. 1, when X-rays are irradiated from the upper part of the couveuse 500 and the test subject P1 is photographed together with the couveuse 500, the intensity of the X-rays differ between the area penetrating the material of the couveuse 500 and the area penetrating the opening 501 (the area without the material of the couveuse 500 interposed). An exemplary X-ray image in such a case is shown in FIG. 3A. The X-rays do not attenuate in the region corresponding to the opening 501; accordingly, the intensity of the X-rays becomes stronger compared to the other regions, that is, the regions in which the material of the couveuse 500 is penetrated, thereby increasing the luminance. Therefore, as shown in FIG. 3A, an artifact EQ1 shaped like the opening 501 is shaped in the X-ray image E1.

Moreover, not limited to when photographing test subjects P1 placed in a couveuse 500, objects different from the test subject may be photographed together with the test subject. As an example thereof, there are cases in which a calibrated object is imaged together with the test subject EP2. The calibrated object is an object in which the size thereof is already known, and by means of photographing the calibrated object together with the test subject, the image of the calibrated object may be regarded as the index for distance in the X-ray image (for example, the distance per 1 pixel). For example, FIG. 3B shows an example of an X-ray image when a test subject EP2 is photographed together with a calibrated object. The X-ray diagnosis device is able to calculate the distance in the X-ray image E2 (for example, the distance per 1 pixel) based on the calibrated object image EQ2 imaged in the X-ray image E2, as shown in FIG. 3B. Furthermore, the calibrated object image EQ2 becomes unnecessary once the distance in the X-ray image E2 is calculated.

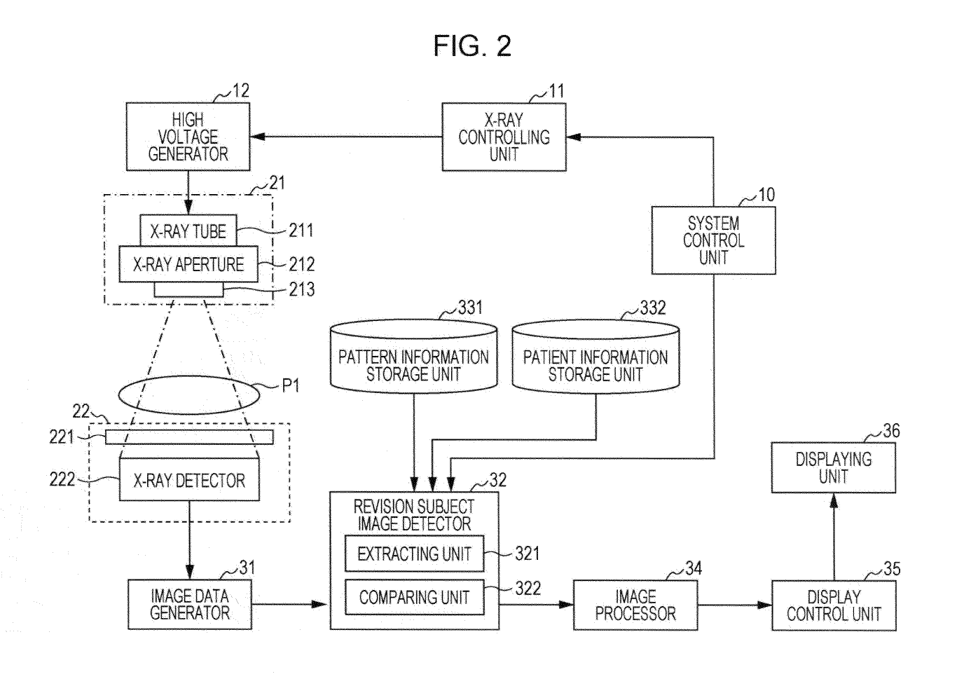

The abovementioned artifact EQ1 shown in FIG. 3A and the calibrated object image EQ2 shown in FIG. 3B may inhibit interpretation of the radiogram. Accordingly, the X-ray diagnosis device related to the present embodiment specifies images such as the artifact EQ1 and the calibrated object image EQ2, etc. in the X-ray image (that is, in the image data), and carries out revision to the image. Hereinafter, the specific configuration of the X-ray diagnosis device related to the present embodiment including the revision operation is described with reference to FIG. 2. FIG. 2 is a block diagram of the X-ray diagnosis device related to the present embodiment. Furthermore, hereinafter, the image showing the artifact EQ1 and the calibrated object image EQ2 in the X-ray image may be referred to as “the image subject to revision.”

As shown in FIG. 2, the X-ray diagnosis device related to the present embodiment is configured by comprising: a system control unit 10, an X-ray controlling unit 11, a high voltage generator 12, an X-ray generating unit 21, a photography platform 22, an image data generator 31, a revision subject image detector 32, a pattern information storage unit 331, a patient information storage unit 332, an image processor 34, a display control unit 35, and a displaying unit 36.

The X-ray generating unit 21 is configured by comprising an X-ray tube 211, an X-ray aperture 212, and a dose area product meter 213. The X-ray tube 211 accelerates electrons output from the filament by means of high voltage, generates X-rays by colliding these electrons into a target, which becomes an anode, and irradiates the outside from an irradiation window. As an example, tungsten may be used as the material of the target. The X-ray aperture 212 is provided on the irradiation window of the X-ray tube 211 and is configured from a plurality of metal blades. The X-ray aperture 212 narrows down the irradiation field to a predetermined size in order prevent the exposure of unnecessary areas other than the observation site to X-rays irradiated from the X-ray tube 211. Moreover, on the output side of the X-ray aperture 212, a compensating filter shaped from acryl, etc., which reduces the X-rays of the predetermined region within the irradiation field by the predetermined amount, may be provided in order to prevent halation.

The dose area product meter 213 detects the dose of X-rays penetrating the X-ray aperture 212. The dose area product meter 213 converts the detected X-ray dose to an electrical charge, and outputs this as the output signal of the dose area substantially proportional to the irradiation intensity, irradiation area, and irradiation time of the X-rays.

The dose area product meter 213 calculates the dose of the area by dividing the calculated X-ray dimension dose of the X-ray irradiation region at a reamer reference position. In other words, the dose area product meter 213 outputs signals showing the X-ray irradiation intensity per unit area as the dimension dose.

The high voltage generator 12 accelerates thermal electrons generated from a cathode, thereby generating high voltage applied between the anode and the cathode. The action of the high voltage generator 12 is controlled by the X-ray controlling unit 11. Specifically, the X-ray controlling unit 11 receives control information exhibiting the X-ray irradiation conditions from the system control unit 10. The X-ray controlling unit 11 generates information exhibiting the X-ray irradiation conditions configured from a tube current, tube voltage, X-ray pulse width, irradiation cycle (rate interval), penetrating intervals, etc. for actuating the high voltage generator 12 based on the control information. The X-ray controlling unit 11 controls the action of the high voltage generator 12 based on the information.

The X-ray detector 222 is configured from, for example, a flat panel detector (FPD, flat-shaped X-ray detector) comprising a plurality of semiconductor detecting elements arranged in a matrix. The X-ray detector 222 detects the intensity of the X-rays irradiated from the X-ray generating unit 21 in the predetermined irradiation field together with the semiconductor detecting element. Furthermore, an X-ray grid that cuts the scattered light of the X-rays penetrating the predetermined area of the test subject P1 may be provided on the surface of the top plate 221 side of the FPD. The X-ray detector 222 converts the intensity of the X-rays detected by each semiconductor detecting element into electrical signals, and outputs them to the image data generator 31 as electric signal. The image data generator 31 is described later. Furthermore, the X-ray detector 222 may be configured from a combination of an X-ray I.I. (image intensifier) and an X-ray TV camera as a substitute for FPD.

The image data generator 31 receives electric signal from the X-ray detector 222. Moreover, for example, as shown in FIG. 3B, when the calibrated object image EQ2 is comprised in the image data, the image data generator 31 calculates the distance (in other words the distance per 1 pixel) in the X-ray image based on the image. For example, FIG. 3B shows the X-ray image E2 when the test subject P2 is photographed together with a calibrated object. As shown in FIG. 3B, the calibrated object image EQ2 is projected upon the X-ray image E2 together with the test subject P2 image. The image data generator 31 calculates the distance per 1 pixel based on the information exhibiting the calibrated object image EQ2 (pixel data) in the image data showing the X-ray image E2. Furthermore, the image data generator 31 may carry out image processing such as concordance adjustment, etc., in advance in order to detect the information exhibiting the calibrated object image EQ2 from among the image data. The image data generator 31 supplements the information exhibiting the calculated distance to the image data.

The image data generator 31 carries out the abovementioned image calculation with respect to the image data, and subsequently outputs the image data that underwent the image calculation to the revision subject image detector 32.

The revision subject image detector 32 receives the image data that underwent image calculation from the image data generator 31. The revision subject image detector 32 is configured by comprising an extracting unit 321 and a comparing unit 322, wherein, it specifies the image subject to revision included in the received image data together with the configuration. The specific actions of the revision subject image detector 32, the extracting unit 321, and the comparing unit 322 are described in the following.

The revision subject image detector 32 carries out edge detection processing with respect to the received image data. As an example of the edge detection processing, the revision subject image detector 32 calculates the gradation variation between adjacent pixels in the image data, and detects the area in which the calculated variation is the predetermined value or more as the edge.

Next, the revision subject image detector 32 outputs the image data with the edge extracted to the extracting unit 321. The extracting unit 321 carries out pattern extraction processing, such as, for example, Hough conversion, etc. with respect to the image data. Hough conversion is a process using a feature extraction method, which is a process of converting normal images on rectangular coordinates to a two-dimensional space of polar coordinates (in the case of detecting straight lines) or converting to a three-dimensional space (in the case of detecting circles) and obtaining the position with the highest frequency from among these, inversely transforming them, and detecting a straight line or circle. Thereby, the extracting unit 321 extracts the circular image or the image shaped with a straight line from among images shown as the region surrounded by the edge. Moreover, the extracting unit 321 calculates the size of the extracted image with the predetermined shape and extracts the image of the predetermined size. Thereby, differentiating between, for example, the area close to the circular shape, such as the head, etc., and the image of the opening 501 is allowed. The extracting unit 321 outputs the received image data and the information exhibiting the extracted image of the predetermined shape to the comparing unit 322.

The comparing unit 322 receives the image data and the information exhibiting the image of the predetermined shape from the extracting unit 321. Moreover, the pattern information generated in advance based on the shape and size of the image subject to revision is stored in the pattern information storage unit 331.

The pattern information is associated with the information exhibiting the classification of the image subject to revision corresponding to the pattern thereof (hereinafter, referred to as classification of the subject image). The classification of the subject image comprises, for example, the information exhibiting the artifact EQ1 shaped from the opening 501 and the information exhibiting the calibrated object image EQ2. The comparing unit 322 reads the pattern information from the pattern information storage unit 331. The comparing unit 322 carries out pattern matching between the information exhibiting the received image of a predetermined shape and the read-out pattern information, and specifies the image corresponding to the pattern information as the image subject to revision.

Furthermore, when there are multiple candidates for the image subject to revision, the pattern information may be generated in advance for each of the candidates and stored in the pattern information storage unit 331. When there is multiple pattern information, for example, it is advisable to associate the pattern information with a study condition (for example, the presence a couveuse 500 as well as a calibrated object). When such configuration is assumed, for example, the comparing unit 322 receives the information showing the study condition from the system control unit 10, and extracts the pattern information associated with the study condition from among the multiple pattern information. The system control unit 10 is described later. Thereby, the comparing unit 322 is not required to carry out pattern matching regarding all pattern information, and reducing the burden from processing related to the pattern matching may be realized.

Moreover, a patient information storage unit 332 exhibiting the physical characteristics of a test subject P1 may be provided and the patient information may be associated with the pattern information. When, for example, there are couveuses 500 of multiple sizes according to the height of the test subject P1, the shape and size of the artifact EQ1 generated for each couveuse 500 may be different. In such cases, the pattern information is generated in advance for each couveuse 500, and the pattern information is associated with the information exhibiting the height included in the patient information. By means of assuming such a configuration, the comparing unit 322 specifies the type of couveuse 500 used based on the patient information of the test subject, and the pattern information corresponding to the specified couveuse 500 may be extracted.

Moreover, the presence of an object photographed together with the test subject such as a calibrated object, etc. (for example, the couveuse 500 and the calibrated object) may be associated with the study information exhibiting the type of study in advance. By means of assuming such a configuration, for example, the comparing unit 322 receives information exhibiting the type of study from the system control unit 10, allowing specification of the object photographed together with the test subject. Thereby, the comparing unit 322 may extract the pattern information corresponding to the specified object. Furthermore, the information associated with the pattern information, such as the patient information, study information, etc., correspond to “attribute information.”

Once the image subject to revision is specified, the comparing unit 322 extracts the classification of the subject image associated with the pattern information used for specification, and associates the information exhibiting the specified image subject to revision with the extracted classification of the subject image. The comparing unit 322 outputs the information exhibiting the image subject to revision associated with the classification of the subject image and the image data to the image processor 34.

Furthermore, the example of actuating the extracting unit 321 and the comparing unit 322 was described above; however, only one among the extracting unit 321 and the comparing unit 322 may be actuated. For example, when the generation of the circular artifact is known in advance, the extracting unit 321 may extract the circular image from among the image data and specify the image as the image subject to revision. In this case, the extracting unit 321 associates the information exhibiting the image subject to revision with the classification of the subject image (for example, the classification showing the artifact) determined in advance, and outputs this to the image processor 34. Moreover, the comparing unit 322 may compare the image shown by the region configured from the edge in the image data and the pattern information and specify the image corresponding to the pattern information as the image subject to revision. The revision subject image detector 32 specifies the image other than the test subject image as the image subject to revision, as in the artifact EQ1 and the calibrated object image EQ2, and outputs the information showing this to the image processor 34.

The image processor 34 receives the information exhibiting the image subject to revision and the image data from the comparing unit 322. The image processor 34 extracts the classification of the subject image associated with the received information exhibiting the image subject to revision, and specifies the classification of the image subject to revision based on the classification of the subject image. Thereby, the image processor 34 specifies whether the image subject to revision is, for example, the artifact EQ1 or the calibrated object image EQ2.

The image processor 34 associates and stores in advance the classification of the subject image and the predetermined image processing. For example, the artifact EQ1 is actualized as the artifact because it has a higher luminosity value than the surrounding region thereof. Thereby, the image processor 34 stores the process of reducing the luminosity value of the image subject to revision (that is, the artifact EQ1) by associating this with the classification of the subject image corresponding to the artifact EQ1. Specifically, the image processor 34 may delete or remove the image subject to revision by making the luminosity value of the image subject to revision the same as the luminosity value of the area adjacent to the image subject to revision. Moreover, when the difference in the luminosity value between the two is the predetermined standard value or more, the image processor 34 may make the luminosity value of the image subject to revision as the value with the predetermined standard value added to the luminosity value of the area adjacent to the image subject to revision. Other than this, by means of changing the luminosity value of both including the luminosity value of the image subject to revision and the luminosity value of the area adjacent to the image subject to revision, the difference in the luminosity value between the two may be made the abovementioned predetermined standard value. Moreover, the calibrated object image EQ2 becomes unnecessary at interpreting radiograms, so it is desirable to delete the image. Therefore, the image processor 34 overwrites the pixel of the image subject to revision with the pixels of the surrounding region, thereby storing the process of deleting the image subject to revision by associating it with the classification of the subject image corresponding to the calibrated object image EQ2. By means of this process, the luminosity value of the image subject to revision becomes similar to the luminosity value of the area adjacent to the image subject to revision. Putting the two abovementioned processes into other words, the image processor 34 revises the image data and reduces the luminosity value of the image subject to revision to the value in which the specified value is added to the luminosity value of the area adjacent to the image subject to revision or less (including the luminosity value of the area adjacent to the image subject to revision). That is, image processing is carried out with respect to the image subject to revision such that the difference in the luminosity value between the luminosity value of the image subject to revision and the luminosity value of the area adjacent to the image subject to revision becomes the specified value or less.

That is, the image processor 34 specifies the classification of the image subject to revision, and subsequently processes the image associated with the specified classification with respect to the image subject to revision from among the image data. Thereby, image processing is carried out according to the classification of the image subject to revision, and the image subject to revision from among the image data is revised.

The image processor 34 may be actuated such that it carries out revision of the image subject to revision, then subsequently carries out the predetermined image processing with respect to the area other than the image subject to revision in the image data. For example, it may be actuated so as to carry out enhancement processing such as unsharp-mask, etc. to the areas other than the image subject to revision. Unsharp-mask is a processing function of enhancing the definition (sharpness) of the image which enhances the color of the outline of the image and the difference in light and shades. The process consists of blurring (unsharpening) the image once, comparing the original image with the blurred image, extracting the difference therebetween, adjusting this and applying it to the original image. An outline enhancing the process of enhancing a high frequency component of the other images may be adopted as a substitute. Moreover, the image processor 34 may be actuated such that the predetermined image processing is carried out on the entire image data including the image subject to revision without limitation to the area other than the image subject to revision. In this manner, in the present embodiment, a process of reducing the luminosity value of the image is carried out before adopting enhancement processing on the image; therefore, enhancement processing is carried out such that the unnecessary images do not interfere, thereby allowing simple interpretation of the radiogram of the image.

Moreover, the image processor 34 should specify the image processing according to the classification of the image subject to revision (classification of the subject image), with the method thereof not limited to those mentioned above. For example, it may be actuated such that the image processor 34 receives the attribute information, the classification of the subject image is specified based on the attribute information, and the image processing corresponding to the classification of the subject image is carried out on the image data.

The image processor 34 outputs the image data that underwent image processing to the display control 35. Upon receiving this data, the display control 35 displays the X-ray image on the display unit 36 based on the image data.

The system control unit 10 configures a core of the control of all systems, receives the X-ray irradiation conditions input by the operator as the conditions for X-ray examination, and controls the action of the X-ray controlling unit 11. Specifically, the system control unit 10 generates control signals based on the X-ray irradiation conditions input by the operator, and controls the action of the X-ray controlling unit 11 by means of the control signals. By means of the control signals, the X-ray controlling unit 11 actuates the high voltage generator 12 and irradiates X-rays from the X-ray generating unit 21.

Moreover, the system control unit 10 may be actuated such that it receives study information (for example, the use of a couveuse 500 and the use of a calibrated object) showing the classification of the study input by the operator and output the study information to the revision subject image detector 32. By means of actuating the system control unit 10 in this manner, the revision subject image detector 32 confirms the use of a couveuse 500 and the use of a calibrated object, allowing reading of the corresponding pattern information.

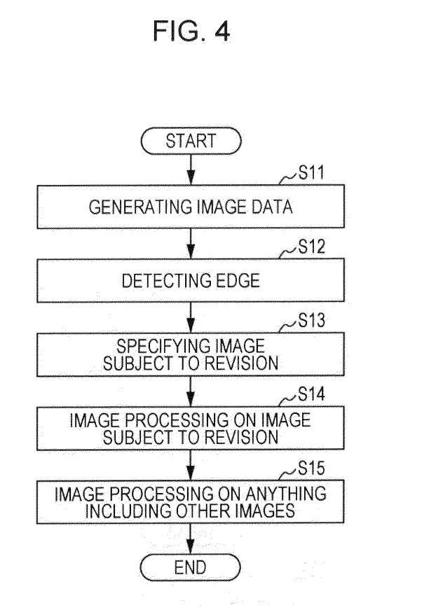

Next, the series of actions of the X-ray diagnosis device related to the present embodiment are described with reference to FIG. 4. FIG. 4 is a flow chart showing the series of actions of the X-ray diagnosis device related to the present embodiment.

(Step S11) The system control unit 10 generates control signals based on the X-ray exposure conditions input by the operator, controlling the action of the X-ray controlling unit 11 by means of the control signals. By means of the control signals, the X-ray controlling unit 11 actuates the high voltage generator 12 and irradiates X-rays from the X-ray generating unit 21.

The X-ray detector 222 detects the intensity of the X-rays irradiated from the X-ray generating unit 21 in the predetermined irradiation field per semiconductor detecting element. The X-ray detector 222 outputs the intensity of the X-ray irradiated from the X-ray generating unit 21 output per semiconductor detecting element to the image data generator 31 upon converting this into electrical signals.

The image data generator 31 receives the image data from the X-ray detector 222, and carries out image calculation on the image data. The image data generator 31 carries out the abovementioned image calculation with respect to the image data, and subsequently outputs the image data that underwent the image calculation to the revision subject image detector 32.

(Step S12) The revision subject image detector 32 receives the image data following image calculation from the image data generator 31. The revision subject image detector 32 carries out edge detection processing with respect to the received image data. As an example of edge detection processing, the revision subject image detector 32 calculates the gradation variation between adjacent pixels in the image data, and detects the area in which the calculated variation is the predetermined value or more as the edge.

(Step S13) Next, the revision subject image detector 32 outputs the image data with the edge extracted to the extracting unit 321. The extracting unit 321 carries out pattern extraction processing, such as, for example, Hough conversion, etc. with respect to the image data. Thereby, the extracting unit 321 extracts the image of the predetermined shape such as the shape configured from straight lines, circular shapes, etc. from among the images shown as the region surrounded by the edge. The extracting unit 321 outputs the image data and the extracted information exhibiting the image of the predetermined shape to the comparing unit 322.

The comparing unit 322 receives the image data and the information exhibiting the image of the predetermined shape from the extracting unit 321. Moreover, the pattern information generated in advance based on the shape and size of the image subject to revision is stored in the pattern information storage unit 331. The pattern information is associated with the information exhibiting the classification of the image subject to revision corresponding to the pattern (hereinafter, the classification of the subject image). The comparing unit 322 reads out the pattern information from the pattern information storage unit 331. The comparing unit 322 carries out pattern matching between the information exhibiting the received image of the predetermined shape and the read-out pattern information, and specifies the image corresponding to the pattern information as the image subject to revision.

After the image subject to revision is specified, the comparing unit 322 extracts the classification of the subject image associated with the pattern information used for specification, and associates the classification of the subject image with the information exhibiting the image subject to revision with the specified image subject to revision. The comparing unit 322 outputs the information exhibiting the image subject to revision associated with the classification of the subject image and the image data to the image processor 34.

(Step S14) The image processor 34 receives the information exhibiting the image subject to revision and image data from the comparing unit 322. The image processor 34 extracts the classification of the subject image associated with the information exhibiting the received image subject to revision, and specifies the classification of the image subject to revision based on the classification of the subject image.

The image processor 34 associates and stores in advance the classification of the subject image and the predetermined image processing. The image processor 34 specifies the classification of the image subject to revision and subsequently carries out the image processing associated with the specified classification with respect to the image subject to revision in the image data. Thereby, the image processing is carried out according to the classification of the image subject to revision, and the image subject to revision in the image data is revised.

(Step S15) The image processor 34 may be actuated such that it carries out revision with respect to the image subject to revision, and subsequently carries out the predetermined image processing with respect to the areas other than the image subject to revision in the image data. For example, it may be actuated such that enhancement processes such as un-sharp mask, etc. are carried out with respect to the areas other than the image subject to revision. Moreover, the image processor 34 may be actuated such that the predetermined image processing is carried out on the entire image data including the image subject to revision without limitation to the areas other than the image subject to revision.

The image processor 34 outputs the image data that underwent image processing to the display control 35. Upon receiving this, the display control 35 displays the X-ray image on the displaying unit 36 based on the image data.

As mentioned above, the X-ray diagnosis device related to the present embodiment specifies images with a predetermined shape as the image subject to revision, and processes images according to the classification of images subject to revision with respect to the image subject to revision thereof. Thereby, revision upon specifying unnecessary images becomes possible during diagnosis of the radiogram, as in, for example, the artifact EQ1 generated due to the opening 501 of the couveuse 500 shown in FIG. 3A, and the calibrated object image EQ2 shown in FIG. 3B.

It should be noted that it is possible to apply the above embodiment even if a cover is provided over the opening 501.

While certain embodiments have been described, these embodiments have been presented by way of example only, and are not intended to limit the scope of the inventions. Indeed, the novel systems described herein may be embodied in a variety of their forms; furthermore, various omissions, substitutions and changes in the form of the systems described herein may be made without departing from the spirit of the inventions. The accompanying claims and their equivalents are intended to cover such forms or modifications as would fall within the scope and spirit of the inventions.

What is claimed is:

1. An X-ray diagnosis device, comprising: an X-ray source irradiating X-rays towards a test subject, an X-ray detector configured to detect an intensity of the X-rays penetrating the test subject, and an image data generator that generates X-ray images based on the intensity of the X-rays detected using the X-ray detector, the X-ray diagnosis device photographing an object different from the test subject together with the test subject, the X-ray diagnosis device, comprising:
an image subject revision detector configured to detect an image of the object or an image shaped based on the shape of the object from among the X-ray images as images subject to revision, and,
an image processor that processes images with respect to the image subject to revision such that the difference between the luminosity value of the image subject to revision and the luminosity value of the area adjacent to the image subject to revision becomes a specified value or less.

2. The X-ray diagnosis device according to claim 1, wherein;
the X-ray diagnosis device comprises a pattern information storage unit that stores pattern information exhibiting the shape, and
the image subject revision detector is configured to compare the pattern information with the image included in the X-ray image, and detect an image coinciding with the pattern information as the image subject to revision.

3. The X-ray diagnosis device according to claim 1, wherein;
the image of the object or the image shaped based on the shape of the object is a circle, and
the image subject revision detector is configured to extract a circular image from the X-ray images to detect the extracted circular image as the image subject to revision.

4. The X-ray diagnosis device according to claim 1, wherein;
the image subject revision detector is configured to extract the circular image or an image shaped with a straight line from among images included in the X-ray images, to compare the pattern information with the extracted images, to detect the image coinciding with the pattern information as the image subject to revision.

5. The X-ray diagnosis device according to claim 2, wherein;
the pattern information storage unit associates and stores in advance attribute information comprising at least one among patient information exhibiting the physical characteristics of the test subject and study information exhibiting a classification of the test towards the test subject with the pattern information, and
the image subject revision detector is configured to extract the pattern information corresponding to the attribute information that is input in advance from the pattern information storage unit, to detect the image coinciding with the extracted pattern information as the image subject to revision.

6. The X-ray diagnosis device according to claim 1, wherein,
the object comprises an opening and is a case for storing the test subject inside,
the image subject revision detector is configured to detect the image subject to revision that is an image with the same shape as the opening from among the X-ray images in which the test subjects placed inside the case are photographed together with the case, and
the image processor is configured to reduce the luminosity value of the image subject to revision to a value with a specified value added to the luminosity value of the area adjacent to the image subject to revision.
